(19) United States
(12) Patent Application Publication
(10) Pub. No.: US 2010/0168748 A1
(43) Pub. Date: Jul. 1, 2010

(54) MORSELIZER

(76) Inventors: Peter G. Knopp, Pleasanton, CA (US); Paul E. Chirico, Campbell, CA (US); Benny M. Chan, San Jose, CA (US); Alison M. Souza, Santa Clara, CA (US)

Correspondence Address: SHAY GLENN LLP, 2755 CAMPUS DRIVE, SUITE 210, SAN MATEO, CA, 94403, US

(21) Appl. No.: 12/504,066

(22) Filed: Jul. 16, 2009

Related U.S. Application Data
(60) Provisional application No. 61/081,308, filed on Jul. 16, 2008.
(60) Provisional application No. 61/121,309, filed on Dec. 10, 2008.

(51) Int. Cl. A61B 17/00

(52) U.S. Cl. 606/79

(57) ABSTRACT

Described herein are bone morcelizers for forming cavities in bone, particularly cancellous bone. In general, these devices include an outer cannulated member coupled to a proximal handle. An inner member may be extended and rotated relative to the outer member in a controlled manner, by operating one or more controls on the handle. The inner member may assume a curved shape relative to the outer member when it is extended.

### CROSS-REFERENCE TO RELATED APPLICATIONS

This application claims the benefit of priority to U.S. Provisional Patent Application Ser. No. 61/081,308, titled “MORSELIZER”, filed on Jul. 16, 2008 and U.S. Provisional Patent Application Ser. No. 61/121,309, titled “MORSELIZER”, filed on Dec. 10, 2008, herein incorporated by reference in their entirety.

This provisional patent application may be related to U.S. patent application Ser. No. 12/025,537, titled “METHODS AND DEVICES FOR STABILIZING BONE COMPATIBLE FOR USE WITH BONE SCREWS”, filed on Feb. 4, 2008. This application may also be related to U.S. patent application Ser. No. 11/468,759, filed on Aug. 30, 2006, which claims the benefit of U.S. Provisional Patent Application Ser. No. 60/713,259, filed on Aug. 31, 2005, and to U.S. Provisional Patent Application Ser. No. 60/916,731, filed on May 8, 2007. All of these applications are incorporated herein by reference in their entirety.

### INCORPORATION BY REFERENCE

All publications and patent applications mentioned in this specification are herein incorporated by reference in their entirety as if each individual publication or patent application was specifically and individually indicated to be incorporated by reference.

### BACKGROUND OF THE INVENTION

Described herein are systems, devices, and methods for treating bone within a skeletal structure. The invention also relates to systems, devices, and methods for forming cavities in cancellous bone, including cancellous bone within vertebral bodies. These devices, systems, and methods may be used to treat vertebral bodies affected by osteoporosis.

Systems and methods for forming, supporting, fusing and expanding bone cavities may include any of the devices described herein, as well as devices and methods adapted for the use with a bone implant, a bone cement and/or a bone filler. For example, at least some of the devices described in U.S. patent application Ser. No. 12/025,537, titled “METHODS AND DEVICES FOR STABILIZING BONE COMPATIBLE FOR USE WITH BONE SCREWS”, filed on Feb. 4, 2008 may be inserted into a cavity formed in a bone using a morcelizer as described herein. The bone cavity may then be expanded, and then filled with bone cements of any appropriate type.

The formation of a bone cavity is often difficult, due to the size constraints, as well as the stresses placed on the devices used to form the cavities in bone. For example, cancellous bone may be accessed through a narrow gap or opening, as described below. However, in order to form a sufficient opening or hole within the cancellous bone, it may be necessary to provide sufficient force (including torque) to compact and/or cut the bone. In many of the devices currently available, the bone compaction/cutting device (e.g., morcelizers) includes a moving distal end that is hinged. Such hinges or hinge points often result in weak regions that may be broken off during use. Breaking of the morcelizer is likely to result in trauma and undesirable outcomes. Furthermore, the handle and grip regions of currently available devices may be difficult to operate. In addition, the tip region of the morcelizer may be insufficient, and may lack orientation.

Thus, it would be desirable to have devices, methods and/or systems for forming a bone cavity, particularly in cancellous bone, so that an implant and/or bone filler, cement or other fluent material may be applied.

Described herein are devices, systems and methods for forming cavities in bone, including bone morcelizers that are extendable from an outer sleeve to assume a curved shape, rotatable, and/or include one or more pre-formed regions for helping compress the bone.

### SUMMARY OF THE INVENTION

Described herein are bone morcelizers for forming cavities in bone, particularly cancellous bone. In general, these devices include an outer cannulated member coupled to a proximal handle. An inner member may be extended and rotate relative to the outer member in a controlled manner, by operating one or more controls on the handle or on a proximal handle attached to the inner rod.

In some variations, the inner member may assume a curved shape relative to the outer member when it is extended. For example, the inner member may be formed of a pre-shaped shape memory material (e.g., a shape memory alloy such as Nitinol). The distal end of the inner member may be formed in a predetermined shape, such as a flattened, spatulate or shovel-shape. The proximal end of the inner member may be coupled to (or may itself form) an inner-rod handle or control knob. For example, the control knob may be configured to be rotated and/or extended from the outer member. The distal end of the outer member may include one or more markings that may be visualized using imaging techniques (e.g. fluoroscopy, etc.). The device may also include a lock for locking the position of the inner member relative to the outer member.

In some variations, the outer cannulated member includes one or more self-expanding cutting struts that may be held in a collapsed form by the inner rod. The struts may include one or more cutting surfaces.

For example, described herein are bone morcelizer devices for forming a cavity in bone, that include: an outer cannulated member having a proximal and a distal end; a handle at the proximal end of the outer cannulated member; an inner morcelizing rod movably positioned within the outer cannulated member, wherein the inner morcelizing rod is configured to assume a curved shape upon exiting the distal end of the outer cannulated member; a cutting surface at the distal end of the inner morcelizing rod; and a lock on the handle configured to lock the inner morcelizing rod relative to the outer cannulated member.

The inner cannulated member may be formed of a shape memory alloy (e.g., Nitinol, or other nickel-titanium alloys). The inner cannulated member may have a wedge-shaped tip. The inner cannulated member may include a blade edge. In some variations, the inner cannulated member is configured to rotate within the outer cannulated member.

The inner cannulated member may include a handle at the proximal end configured to allow manipulation of the inner rod. The handle may include a grip (e.g., a finger grip) and may be a knob, lever, etc. This proximal handle on the inner rod may be rotatable and/or extendable. For example, the control for the proximal handle may be threaded, allowing controlled advancement/retraction of the inner rod. These threads may also interact with the lock or locking mechanism to prevent it from advancing or withdrawing in an uncontrolled manner.

In some variations, the curved shape of the inner cannulated member is configured to be at a right angle to the outer cannulated member.

Also described herein are bone morcelizer devices for forming a cavity in bone that include: an outer cannulated member having a proximal and a distal end; a plurality of struts at the distal end region of the outer cannula configured to self-expand into a bow shape for cutting; a handle at the proximal end of the outer cannulated member; an inner rod movably positioned within the outer cannulated member and coupled to the distal end region of the outer cannulated member, distal to the struts, wherein the inner rod is configured to apply force to maintain the struts in a collapsed configuration; and a lock on the handle configured to lock the inner rod relative to the outer cannulated member.

A morcelizer device may also include one or more cutting surfaces on the struts. For example, a strut may include a cutting surface that is oriented radially outward from the outer cannulated member, to the side of the outer cannulated member, and/or radially inward from the outer cannulated member. A cutting surface includes a sharp surface such as a blade/knife-edged surface, a surface including an electrosurgical cutting element (e.g., an electrode configured to apply RF or thermal energy for cutting), or the like.

As mentioned above, the morcelizer device may also include a handle on the proximal end of the inner rod. The lock may also be located on the proximal handle of either the outer cannula or the inner rod.

In some variations, the morcelizer devices also include a tissue-penetrating distal end. For example, the inner rod may include a tissue-penetrating distal end, and/or the outer cannulated member may include a tissue penetrating distal end. Alternatively, in some variations, the distal end is configured so that it does not penetrate tissue (e.g., it is blunt or substantially atraumatic).

Also described herein are methods of morcelizing bone and/or other tissues. For example, described herein are methods of forming or expanding a cavity in a bone, the method including the steps of: inserting a bone morcelizing device having an outer cannulated member and an inner morcelizing rod into a bone; extending the inner morcelizing rod from the distal end of the outer cannulated member so that the inner morcelizing rod assumes a curved shape; and rotating the inner morcelizing rod to cut or compress bone.

The methods may also include the step of locking the inner rod relative to the outer member.

In some variations the step of rotating the inner morcelizing rod comprises locking the inner rod to the outer member and grasping a handle connected to the proximal end of the outer member.

### BRIEF DESCRIPTION OF THE DRAWINGS

FIG. 1 shows one variation of a morcelizer in which the inner member (inner shape memory rod) is retracted proximally into the outer member.

FIGS. 2-6 illustrate extension of the inner member relative to the outer member for a morcelizer such as the morcelizer shown in FIG. 1.

FIGS. 7-9 illustrate rotation of the inner member relative to the outer member for a morcelizer such as the morcelizer shown in FIG. 1.

FIGS. 10A and 10B illustrate another variation of the distal end of a morcelizer.

FIGS. 11A-11C illustrate variations of self-expanding morcelizers having cutting edges.

FIG. 12A shows another variation of a morcelizer as described herein, having a distal morcelizing region as shown in FIGS. 10A and 10B. FIG. 12B shows the distal end of the morcelize of FIG. 12A in the collapsed configuration.

### DETAILED DESCRIPTION OF THE INVENTION

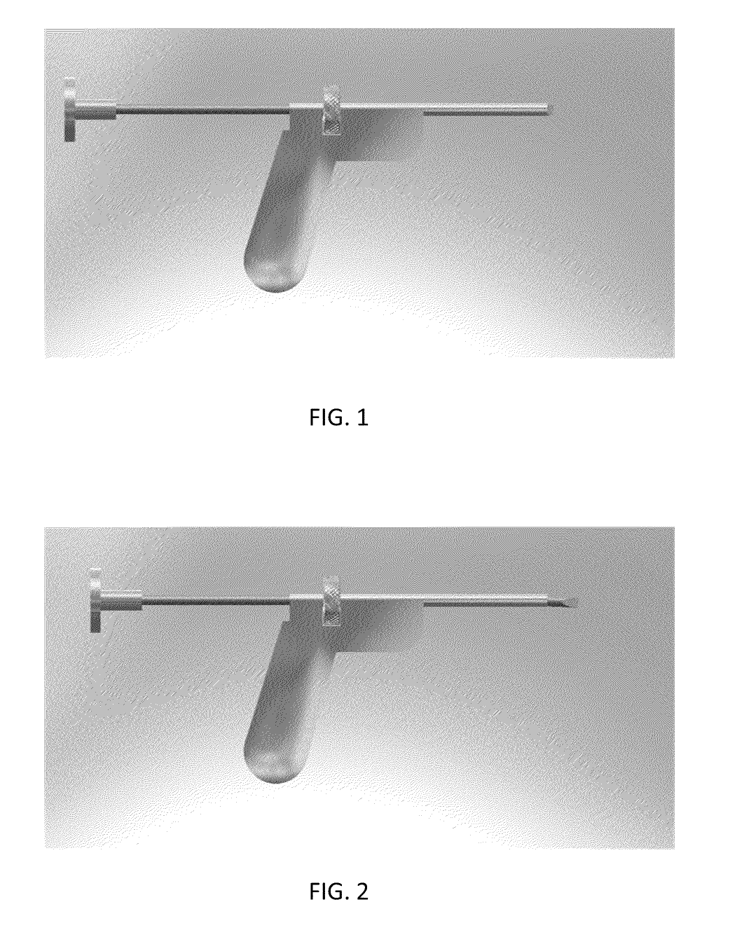

FIG. 1 illustrates one variation of a morcelizer. In general, this device includes an outer cannulated member that is connected (rigidly) to a handle, an inner member that is movable and lockable with respect to the inner member. At least the distal region of the inner member may be pre-shaped so that it assumes a curved or bent configuration when exiting the device. The device typically also includes one or more locks that may securely lock the inner member in position relative to the outer member. For example, in FIG. 1, the lock is a thumbwheel that can be rotated to lock the inner member in position relative to the outer member.

The inner member may be configured to extend from the outer member so that it can assume a curved shape. The inner member (rod) can be rotated independently of the outer member and handle, or it can be locked so that moving the handle will move the inner member, allowing formation of a cavity.

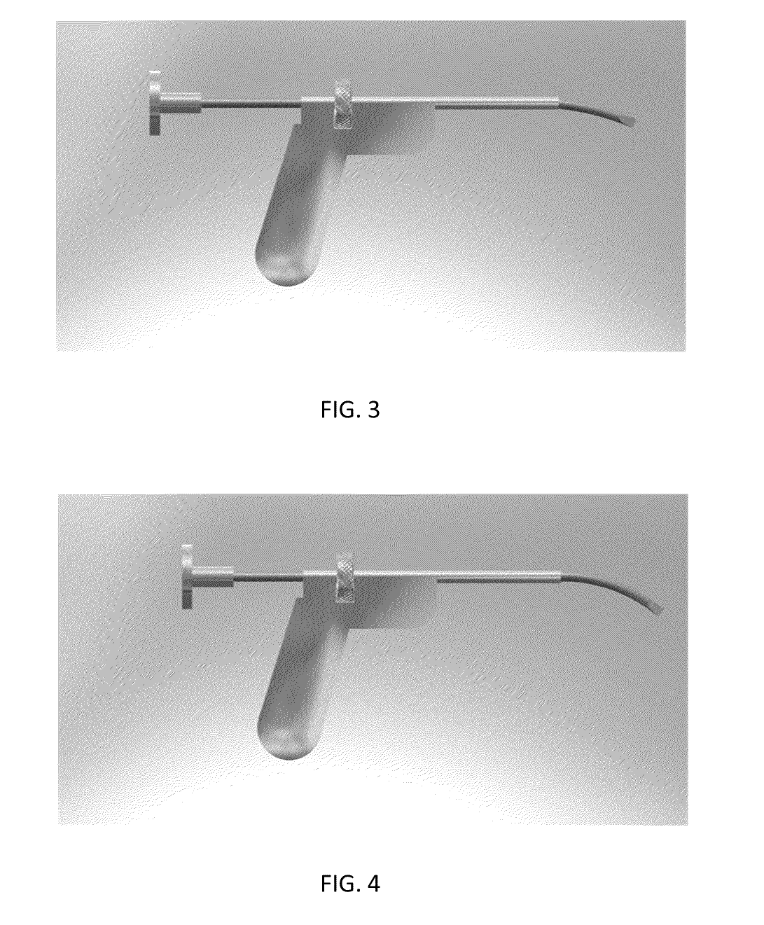

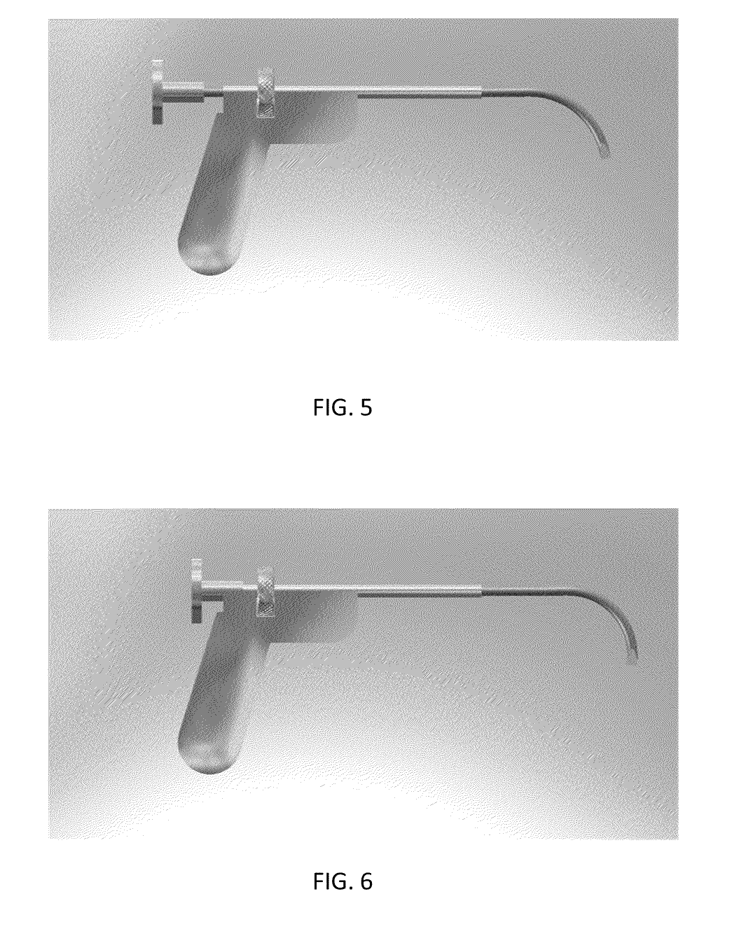

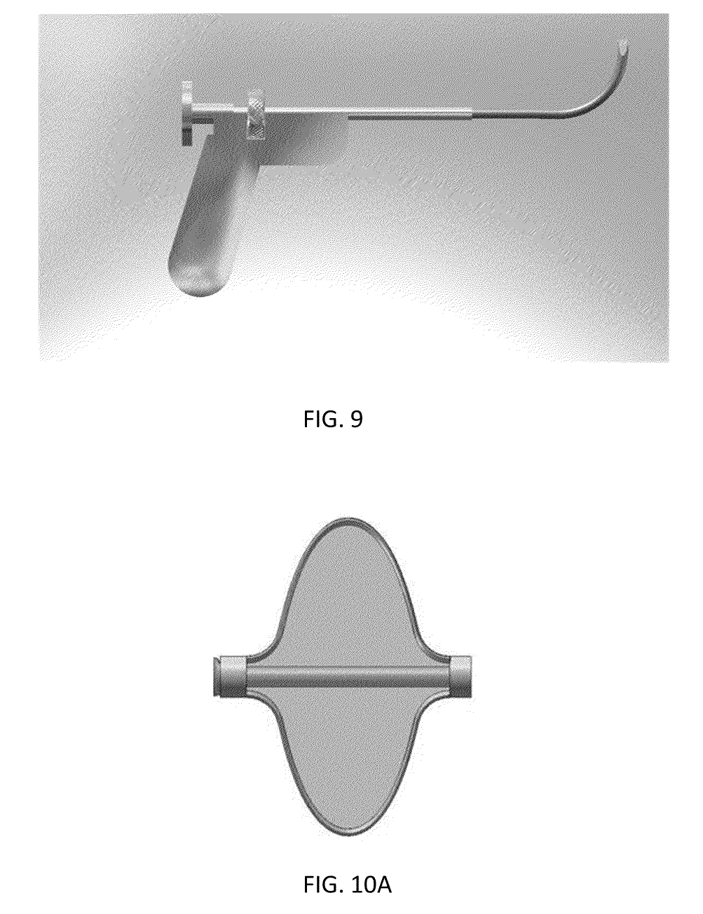

In the example, shown in FIGS. 1-9, the components illustrated are roughly scaled so that the inner rod is approximately 3.8 mm diameter. The curvature of the rod when fully extended may be less than that illustrated.

In operation, the inner member (morcelizer rod) may be withdrawn into the outer member (either completely or partially) and the distal end of the device may be inserted into a bone. For example, a drill may be used to from an opening into a bone. After the distal end is placed in the bone, the inner member may be extended to widen or expand the cavity in the bone. The extent to which the inner member is extended (and thus the exposed curvature of the bone) may determine the size (e.g., width) of the cavity formed. In some variations, rather than the rod being extended to cause the cutting action, the tube can be withdrawn. This means may be preferable since the starting point of the cut would be more easily visualized.

The distal end of the inner member (morcelizing rod) may include one or more cutting surfaces. The cutting surface shown is a simple two-face bevel aligned with the longitudinal axis of the rod. The surface could have different profiles, for example, multiple cutting facets, curvilinear bevel, cupped (similar to curette), and different orientations, for example, orthogonal to or at another angle to the axis. Cutting surfaces that are available in more than one configuration could make the device more broadly useful.

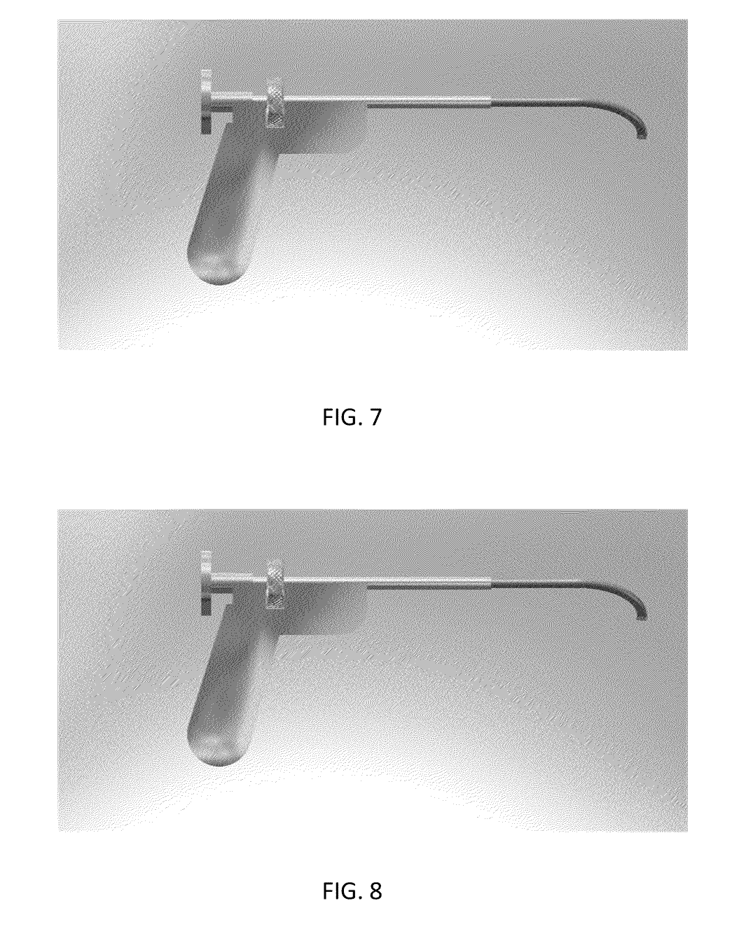

The morcelizing rod may be rotated to further enlarge a cavity in bone (especially cancellous bone). For example, the cutting rod can be rotated directly (as shown in FIG. 7-9), or indirectly by locking the morcelizing rod and the outer cannulated member with handle, then rotating the handle.

In some variations, the rod is a tube, rather than a solid rod. For example, the morcelizing rod may be cannulated. This may be used a biopsy or delivery device.

Although the morcelizing rod may be a single piece (e.g., of pre-biased shape memory alloy), it could alternatively be composed of more than one section and/or be made from more than one material to benefit handling, performance, and cost.

In some variations the cutting surface at the distal end of the morcelizing rod is replaceable or removable. For example, the distal cutting surface could be threaded onto or otherwise connected to the distal end of the rod; thereby making it replaceable.

In some variations, the device may include electronic components that allow the device to electrically cut or cauterize tissue. Thus, the morcelizing device may be configured as an electrocautery device.

In some variations, the device may include one or more wires at preferably radial distances from the rod that can be extended into surrounding tissue to stabilize the rod's cutting action.

### Expandable Morcelizer

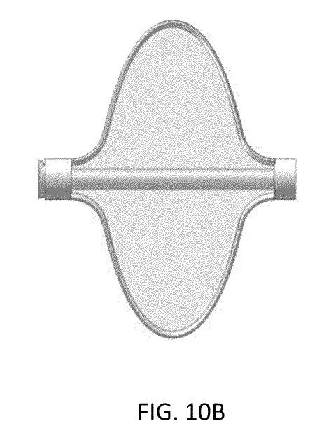

FIGS. 10A and 10B illustrate another variation of a morcelizer configured to expand from a first (e.g., linear) delivery configuration into a cutting configuration. In this variation, the device includes two expandable struts that may be secured at either end to a collar. The struts may be expanded from a collapsed configuration into a curved configuration to form a cutting plane, as indicated in FIG. 10A (shaded area). Although two struts are shown (arranged opposite from each other to form a plane), they may be arranged in any appropriate orientation, and more than two struts may be used. The struts form a cutting zone that is determined by the nominal size of the expanded device (indicted in the gray shading in FIG. 10A). After being inserted (e.g., into cancellous bone), the device may be expanded to cut through the bone as the struts expand. FIG. 10B shows the device in the expanded configuration.

The struts may be adapted for cutting. In some variations the struts are pre-biased in the expanded shape. Thus, the struts may be formed of a shape memory material, such as a shape memory alloy (e.g., Nitinol). The struts may include a cutting edge or surface, e.g., along the outer edge (toward the direction of expansion). In some variations, the struts include serrated or sharp edges facing the direction of expansion. In some variations, the struts include side-cutting edges, that allow cutting should the device be rotated. An inner cutting edge, allowing cutting of the device when collapsing it struts may also be included.

Expandable morcelizers such as the one shown in FIG. 10 may also be included behind a sharp or cutting distal tip. For example, the device may include a sharp distal tip that can be used to drive the device into the tissue, where it can be inserted to position the expandable struts.

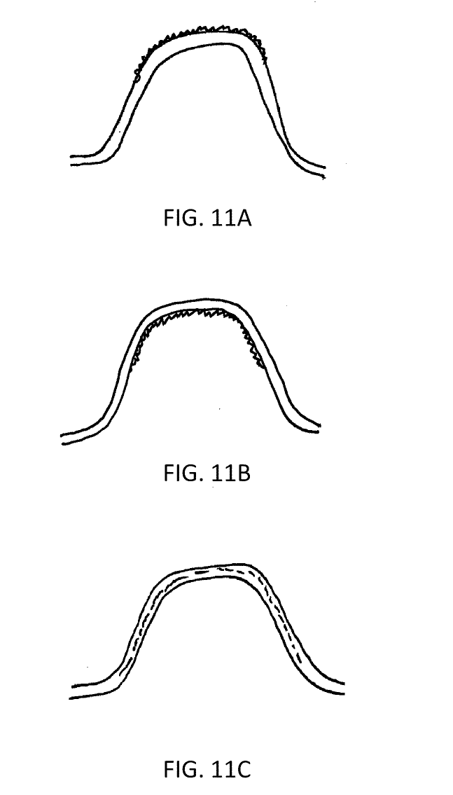

In some variations, the struts extend from an inner member that is surrounded by an outer member, rather than extending from an outer member with an inner member that can hold the struts collapsed or expanded, as shown in FIGS. 10A and 10B. Thus, in some variations the outer member may be a cannula from which an inner rod extends, similar to the embodiment shown and described above. The inner rod may include a pre-biased distal end (e.g., formed of a shape memory alloy) that expands outwards as it is extended from the outer cannula, e.g., by pushing it out of the outer cannulated member. Thus, the struts formed at the distal end of the inner rod of the device may have pre-biased bow shapes (such as the one shown in FIGS. 10 and 11) that can be compressed or collapsed as the inner rod is drawn back into the outer member.

The morcelizer devices shown in FIGS. 10A and 10B include an outer member that includes two bow-shaped struts (e.g., gradually increasing curving upwards, plateauing, then curving downwards, as shown. Other variations may include more than two struts. In some variations the device is pre-biased so that the struts are self-expanding into the expanded shape (shown in FIGS. 10A and 10B). The device is collapsed (or held in the collapsed state) by applying a force across the struts (e.g., pulling the distal and proximal ends of the struts) to flatten them in the delivery (rod-shaped) configuration. The inner rod may be used to apply force. For example, the inner rod may extend proximally to distally within the outer member including the struts. Applying force distally relative to the outer member (or applying force proximally relative to the inner rod) may hold the struts in the collapsed configuration.

Such a pre-biased, self-expanding device may realize significant and unexpected advantages over devices that require the application of force by the user to expand them. Self-expansion may allow for a quick and relatively powerful expansion within small body regions. Further, the addition of force applied by the user can help in expanding the device.

A device including the self-expanding struts shown in FIGS. 10A and 10B may also include a lock at the proximal end, similar to the lock shown in the variation of FIG. 1. In this example, the lock is rotatable (though any appropriate actuation mechanism may be used) to secure the inner rod relative to the outer cannulated member. Similarly, the variation of the distal end shown in FIGS. 10A and 10B (which may have a proximal control/handle similar to that shown in FIG. 1) may include a lock that locks the inner rod in position, holding the device and preventing further self-expansion or accidental collapse.

As mentioned, the struts of the morcelizer may include one or more cutting edges. FIGS. 11A-11C illustrate different cutting edges. For example, FIG. 11A shows one variation of a strut in the expanded configuration having a cutting surface along the outer (e.g., axially outward facing) edge. Although the cutting edge shown in FIG. 11A is a serrated, sharp cutting edge, any appropriate cutting edge may be used. For example, the cutting edge may be a blade or knife edge. In some variations the cutting edge includes one or more electrodes for applying RF energy to cut tissue. FIG. 11B shows another variation of a strut having a cutting edge along the axially inwardly facing edge. Similarly, FIG. 11C shows a strut having a cutting edge along the side-facing edge of the strut. All or a portion (e.g., the central portion) of the strut may include a cutting edge. In some variations more than one edge or face of the strut may include a cutting surface. Thus, the strut may be configured to cut as it expands, as it collapses, as it is rotated, or some combination thereof.

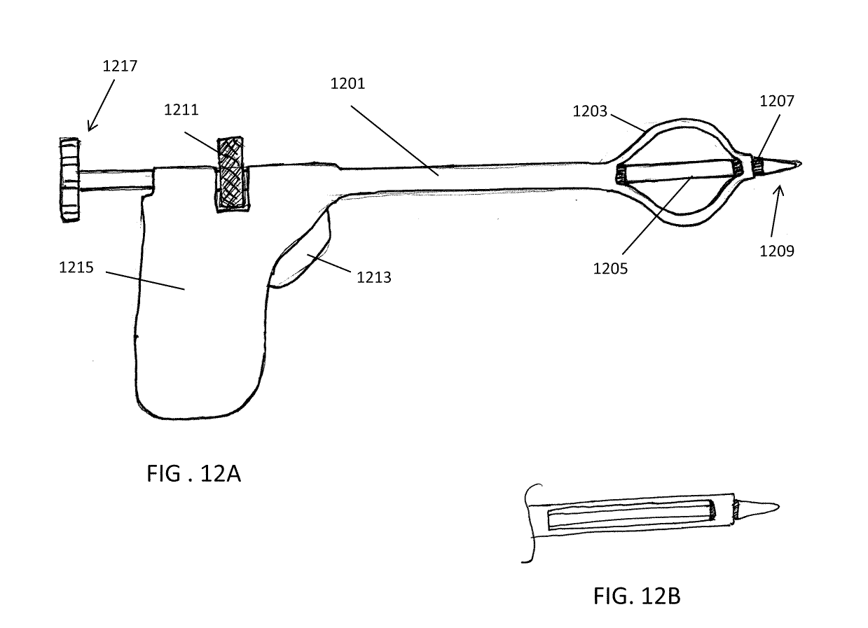

FIGS. 12A and 12B illustrate one variation of a morcelizer including a plurality of self-expanding struts. In FIG. 12A, the morcelizer includes an outer cannula 1201 that includes a handle 1215 at the proximal end and a pair of self-expanding struts 1203 at the distal end. These struts may include one or more cutting edges (not shown). The distal end shown in FIG. 12B illustrates the distal end of the morcelizer shown in FIG. 12A in a collapsed configuration.

An inner, force-applying rod 1205 passes within the cannulated outer member 1201, and is coupled 1207 to the distal end region of the outer cannula. For example, the distal end region and the inner rod may be coupled together by a weld, or by a removable connection.

The distal end of the device may also be tissue penetrating 1209. In this example, the distal end of the rod comprises the distal end of the inner rod 1205, which includes a tapered (and may be pointed) end 1209. The inner rod is also coupled to a locking mechanism 1211, which may be similar to the locking mechanism previously described. In general, this lock may prevent the inner rod 1205 from sliding axially to allow further expansion of the struts (or to contract the struts). The lock may be configured so that it permits rotation of the outer member with struts relative to the inner rod. The lock may be controlled (e.g., engaged/disengaged) by a control such as a trigger 1213. The inner rod 1205 may also include a proximal handle region 1217 that can be used to rotate and/or advance or withdraw the inner rod, and thereby allow or prevent self-expansion of the struts.

In some variations the device may be configured to allow release of the restraining force applied by the inner rod, so that the struts are allowed to freely self-expand. For example, the trigger or control may be configured to release the lock or any inhibition of the inner rod, allowing the self-expansion of the struts.

In some variations the inner rod is coupled to the proximal end of the struts, rather than the distal end. The distal end of the struts may be continuous with the rest of the outer cannula). This allows the struts to expand/contract without extending/withdrawing the distal end of the device. Thus, in operation, the distal end of the device may be inserted in position, and held at this distal position while the struts are expanded. Thus, the distal end does not foreshorten. In this variation, the outer cannula may include a cut-out region into which the struts are positioned, so that the distal end of the struts can be coupled to the distal end region of the outer cannula.

In any of these variations, the self-expanding struts may be formed as part of the outer cannula (e.g., cut-outs of the cannula), or they may be attached or affixed to the outer cannula. Thus the outer cannula and/or inner cannula may be made of other materials, and affixed (e.g., welded, crimped, or otherwise attached to) the struts.

In operation the device may be controlled by a handle that allows one end (e.g., the proximal end) of the expandable struts to be moved distally or proximally so that the device can expand/collapse. This may be accomplished by securing the distal end of the expandable struts to a rod or cannula that may concentrically slide within another cannula secured to the proximal end of the expandable struts. The device may be hollow (e.g., the central rod or cannula) to allow passage of material, visualization, or the like. In some variations, the device may be delivered over another device (e.g., guidewire). For example, the sharp distal end may be secured to a rod or wire that passes through this central passageway. Thus, the device may be exchangeable over other devices.

The expandable struts may be activated to expand automatically or manually. As mentioned, the struts may be pre-biased in the expanded shape, or they may be pre-biased in the collapsed shape.

In general, expandable morcelizers such as those described above must be configured so that they do not readily break, particularly when expanded. If this occurs, the device is likely to damage the patient upon being withdrawn. Thus, in some variations the morcelizers are adapted to prevent breakage, including breakage from misuse. For example, the devices should not be rotated in the expanded configuration, and may include a lock to prevent rotation when expanded. For example, the device may include a clutch on the handle of the device that prevents it from being rotated by rotating the handle when expanded. In some variations, the device may include a rotation joint between the expandable struts and the handle that prevents rotation of the handle from translating into rotation of the expanded struts. In some variations the struts are reinforced to further prevent breakage.

The struts may expand into a symmetric shape, (as shown) or an asymmetric shape (e.g., biased towards one side or the other). The struts may be formed of any appropriate material, as mentioned, including Nitinol, steel, or other alloys. The struts may be expanded to any degree desired. In some variations, the morcelizer device may include a gauge or other indicator that shows how far the expandable device has been expanded.

The methods described herein outline only one example of the morcelizing devices described herein, and additional variations are within the scope of the invention. While embodiments of the present invention have been shown and described herein, such embodiments are provided by way of example only. Thus, alternatives to the embodiments of the invention described herein may be employed in practicing the invention. The exemplary claims that follow help further define the scope of the systems, devices and methods (and equivalents thereof).

What may be claimed is:

1. A bone morcelizer device for forming a cavity in bone, the device comprising:
an outer cannulated member having a proximal and a distal end;
a handle at the proximal end of the outer cannulated member;
an inner morcelizing rod movably positioned within the outer cannulated member, wherein the inner morcelizing rod is configured to assume a curved shape upon exiting the distal end of the outer cannulated member;
a cutting surface at the distal end of the inner morcelizing rod; and
a lock on the handle configured to lock the inner morcelizing rod relative to the outer cannulated member.

2. The device of claim 1, wherein the inner cannulated member comprises a shape memory alloy.

3. The device of claim 1, wherein the inner cannulated member has a wedge-shaped tip.

4. The device of claim 1, wherein the inner cannulated member is configured to rotate within the outer cannulated member.

5. The device of claim 1, wherein the inner cannulated member comprises a handle at the proximal end configured to allow manipulation of the inner rod.

6. The device of claim 1, wherein the curved shape of the inner cannulated member is configured to be at a right angle to the outer cannulated member.

7. A bone morcelizer device for forming a cavity in bone, the device comprising:
an outer cannulated member having a proximal and a distal end;
a plurality of struts at the distal end region of the outer cannula configured to self-expand into a bow shape for cutting;
a handle at the proximal end of the outer cannulated member;
an inner rod movably positioned within the outer cannulated member and coupled to the distal end region of the outer cannulated member, distal to the struts, wherein the inner rod is configured to apply force to maintain the struts in a collapsed configuration; and
a lock on the handle configured to lock the inner rod relative to the outer cannulated member.

8. The device of claim 7, further comprising one or more cutting surfaces on the plurality of struts.

9. The device of claim 8, wherein the cutting surface is oriented radially outward from the outer cannulated member.

10. The device of claim 8, wherein the cutting surface is oriented to the side of the outer cannulated member.

11. The device of claim 8, wherein the cutting surface is oriented radially inward from the outer cannulated member.

12. The device of claim 8, further comprising a handle on the proximal end of the inner rod.

13. The device of claim 8, further comprising a tissue-penetrating distal end.

14. A method of forming or expanding a cavity in a bone, the method comprising:
inserting a bone morcelizing device having an outer cannulated member and an inner morcelizing rod into a bone;
extending the inner morcelizing rod from the distal end of the outer cannulated member so that the inner morcelizing rod assumes a curved shape; and
rotating the inner morcelizing rod to cut or compress bone.

15. The method of claim 2, further comprising locking the inner rod relative to the outer member.

16. The method of claim 2, wherein the step of rotating the inner morcelizing rod comprises locking the inner rod to the outer member and grasping a handle connected to the proximal end of the outer member.
